SEQUENCE LISTING (5 sequences)

SEQ ID NO: 1 - DNA, 660 bp, Cannabis sp.
  source 1..660: mol_type = genomic DNA
atggacacaccagaagaaggaatcggaatcaaaatctacaacacaaccccacaagacgacatagaatcatcctcactaccttaccaccctccatccacggcagttccaggacaaggtcgaaagcgacgcgccgtcgcaaagggcgtgcaaaagacgctctcaaaaacatcaatgctcgcaaattttctcccaacgggaactcttctgactttcgagatggttcttccgtcaatctacggtaacggcgagtgttcccctgtttcaaccaccatgatgcacgtcttattgggtctctgttctctctcctgcttcttcttccatttcaccgacagtttccggggaccggacgggaaggtgtattacgggtttgttaccactaagggtttggctgtgtttaagcctggattggatgttgaagttcccaaagatgagaggtataaattgggtgtcactgattttgttcatgctcttatgtctgttttggttttcacggcgatcgctttttccgatcaccggataactgactgtgtttttcctggacatgagaaagctatggatgaagttatggagagttttcctttgatggttgggattgtttgtagtggcttgtttcttgtttttccaaatactcgttatggtattggatgcatggctacttaa

SEQ ID NO: 2 - DNA, 669 bp, Cannabis sp.
  source 1..669: mol_type = genomic DNA
atggacacaccagaagaaggaatcggaatcaaaatctacaacacaaccccacaagacgacatagaatcatcctcactaccttaccaccctgatcagcctccatccacggcagttccaggacaaggtcgaaagcgacgcgccgtcgcaaagggcgtgcaaaagacgctctcaaaaacatcaatgctcgcaaattttctcccaacgggaacccttctgactttcgagatggttcttccgtcaatctacggtaacggcgagtgttcccctgtttcaaccaccatgatgcacgtcttattgggtctctgttctctctcctgcttcttcttccatttcaccgacagtttccggggaccggacgggaaggtgtattacgggtttgttaccactaagggtttggctgtgtttaagcctggattggatgttgaagttcccaaagatgagaggtataaattgggtgtcactgattttgttcatgctcttatgtctgttttggttttcacggcgatcgctttttccgatcaccggataactgactgtgtttttcctggacatgagaaagctatggatgaagttatggagagttttcctttgatggttgggattgtttgtagtggcttgtttcttgtttttccaaatactcgttatggtattggatgcatggctacttaa

SEQ ID NO: 3 - DNA, 669 bp, Cannabis sp.
  source 1..669: mol_type = genomic DNA
atggacacaccagaagaaggaatcggaatcaaaatctacaacacaaccccacaagacgacatagaatcatcctcactaccttaccaccctgatcagcctccatccacggcggttccgggacaaggtcgaaagcgacgcgccgtcgcaaagggcgtgcaaaagacgctctcaaaaacatcaatgctcgcaaattttctcccaacgggaactcttctgactttcgagatggttcttccgtcaatctacggtaacggcgagtgttcccctgtttcaaccaccatgatgcacgtcttattgggtctctgttctctctcctgcttcttcttccatttcaccgacagtttccggggaccggacgggaaggtgtattatgggtttgttaccactaagggtttggctgtgtttaagcctggattggatgttgaagttcccaaagatgagaggtataaattgggtgttactgattttgttcatgctcttatgtctgttttggttttcacggcgatcgctttttccgatcaccggataactgactgtgtttttcctggacatgagaaagctatggatgaagttatggagagttttcctttgatggttgggattgtttgtagtggcttgtttcttgtgtttccaaatactcgttatggtattggatgcatggcaacttaa

SEQ ID NO: 4 - DNA, 657 bp, Cannabis sp.
  source 1..657: mol_type = genomic DNA
atggacacaccagaaggaatcggaatcaaaatctacgacacaaccccacaagacgacatagaatcatcctcactaccttaccaccctccatccacggcagttccaggacaaggtcgaaagcgacgcgccgtcgcaaagggcgtgcaaaagacgctctcaaaaacatcaatgctggcaaattttctcccaacgggaacccttctgactttcgagatggttcttccgtcaatctacggtaacggcgagtgttcccctgtttcaaccaccatgatgcacgtcttattgggtctctgttctctctcctgcttcttcttccatttcaccgacagtttccggggaccggacgggaaggtgtattacgggtttgttaccactaagggtttggctgtgtttaagcctggattggatgttgaagttcccaaagatgagaggtataaattgggtgttactgattttgttcatgctcttatgtctgttttggttttcacggcgatcgctttttccgatcaccggataactgactgtgtttttcctggacatgagaaagctatggatgaagttatggagagttttcctttgatggttgggattgtttgtagtggcttgtttcttgtgtttccaaatactcgttatggtattggatgcatggctacttaa

SEQ ID NO: 5 - DNA, 666 bp, Cannabis sp.
  source 1..666: mol_type = genomic DNA
atggacacaccagaaggaatcggaatcaaaatctacgacacaaccccacaagacgacatagaatcatcctcactaccttaccaccctgatcagcctccatccacggcagttccgggacaaggtcgaaagcgacgcgccgtcgcaaagggcgtgcaaaagacgctctcaaaaacatcaatgctcgcaaattttctcccaacgggaacccttctgactttcgagatggttcttccgtcaatctacggtaacggcgagtgttcccctgtttcaaccaccatgatgcacgtcttattgggtctctgttctctctcctgcttcttcttccatttcaccgacagtttccggggaccggacgggaaggtgtattatgggtttgttaccactaagggtttggctgtgtttaagcctggattggatgttgaagttcccaaagatgagaggtataaattgggtgttactgattttgttcatgctcttatgtctgttttggttttcacggcgatcgctttttccgatcaccggataactgactgtgtttttcctggacatgagaaagctatggatgaagttatggagagttttcctttgatggttgggattgtttgtagtggcttgtttcttgtgtttccaaatactcgttatggtattggatgcatggctacttaa

(19) United States
(12) Patent Application Publication
(10) Pub. No.: US 2023/0397547 A1
(43) Pub. Date: Dec. 14, 2023

(54) Method to Produce Cannabis Haploid Plants and Consequent Spontaneous or Chemically Induced Haploid/Di-Haploid in a Single Generation

(71) Applicant: Haplotype Holdings Inc., Las Vegas, NV (US)

(72) Inventor: Andrew Phengrasmy, Las Vegas, NV (US)

(21) Appl. No.: 18/333,056

(22) Filed: Jun. 12, 2023

Related U.S. Application Data
(60) Provisional application No. 63/366,300, filed on Jun. 13, 2022.
(60) Provisional application No. 63/387,719, filed on Dec. 16, 2022.

(51) Int. Cl. A01G 22/15; C12Q 1/6895; A01H 6/28; A01H 5/02; A01H 1/08; A01H 1/04

(52) U.S. Cl. CPC A01G 22/15; C12Q 1/6895; A01H 6/28; A01H 5/02; A01H 1/08; A01H 1/045; C12Q 2600/156

(57) ABSTRACT

Provided herein are methods of producing a Cannabis haploid/di-haploid plant in a single generation. The methods comprise (a) obtaining pollen comprising a mutation in DMP8; (b) using the pollen to pollinate a Cannabis plant; (c) collecting a seed from the pollenated Cannabis plant; (d) identifying putative haploid Cannabis plants from the seed; and (e) producing a Cannabis haploid/di-haploid plant in a single generation.

### CROSS-REFERENCE TO RELATED APPLICATIONS

This application claims the benefit of U.S. Provisional Patent Application No. 63/366,300 filed Jun. 13, 2022, and U.S. Provisional Patent Application No. 63,387,719 filed Dec. 16, 2022, each of which is incorporated by reference herein in its entirety.

### REFERENCE TO AN ELECTRONIC SEQUENCE LISTING

The contents of the electronic sequence listing (070500_1US3.xml; Size: 8,746 bytes; and Date of Creation: Jun. 12, 2023) is herein incorporated by reference in its entirety.

### BACKGROUND

Importance of haploid induction: A critical issue that has challenged plant breeders over the years is how to fix the genetic pool once an interesting gene combination has been obtained. Double haploid plants can be used to rapidly fix genetic information (Jacquier et al., “Maize in Planta Haploid Inducer Lines: A Cornerstone for Doubled Haploid Technology,” Doubled Haploid Technology 2288:25-48 (2021)). A first step in this process is the generation of haploid lines that, after chromosome doubling (e.g., colchicine treatment) (Kurtz et al., “Production of Tetraploid and Triploid Hemp,” Hort Science 55(10):1703-7 (2020)) will give homozygous, stable plants that carry the desired traits (Parsons et al., “Polyploidization for the Genetic Improvement of Cannabis sativa,” Frontiers in Plant Science, doi.org/0.3389/fpls.2019.00476 (2019)).

Obtaining homozygous doubled haploid lines has revolutionized crop breeding to (1) accelerate the selection and fixation of desired traits among genetic segregates; (2) develop immortal molecular mapping populations; (3) produce elite homozygous (equivalent to inbred lines) for F1 hybrid production; and (4) fix exclusive important traits of an already existing elite genotype (fix genetic information) (McMahon, “Double Haploid Induction Speeds Up Plant Breeding Process,” Syngenta Thrive, Vol: Summer (2017)).

Conventional breeding of vegetative propagated crops, including Cannabis utilizes the creation of many generations of selfings and backcrosses to establish elite clonal seed propagated homozygous lines derived from the vegetative propagated plants. Doubled haploid production enables the fixation of recombinant haplotypes in elite Cannabis lines within two generations. This allows the production, distribution and use in production, of seed propagated clonal material, genetically equivalent to any original vegetatively propagated elite commercial line. This dramatically reduces both time and costs associated with the development of true breeding seed propagated lines.

Important discoveries in the development of haploid induction: Historically, haploid embryos have been obtained with three different technological strategies. The technological strategies include: (1) use of in vitro culture of haploid cells, usually anther culture, which is the most commonly used method (Jacquier et al., “Puzzling Out Plant Reproduction by Haploid Induction for Innovations in Plant Breeding,” Nature Plants 6:610-9 (2020)); (2) use of in vivo interspecific hybridization or hybridization between very diverse intraspecific lineages; and (3) manipulation of specific genes involved in gamete recognition and successful sporophyte development after fertilization. At least five of such genes have thus far been identified (a) the phospholipase 1A locus, also known as Matrilineal; (b) CENH3 alternate centromeric histone proteins; (c) MSi2 Masashi, RNA binding protein; (d) DMP8, a sperm specific 4-span membrane protein; and (e) ROS mediating protein, ZmPOD65 which appears to be involved in the mechanism of Matrilineal (Jiang et al., “A reactive oxygen species burst causes haploid induction in maize,” Molecular Plant 15(6):943-55 (2022)).

Although in vitro haploid production by another culture is widely used (Jacquier et al., “Puzzling Out Plant Reproduction by Haploid Induction for Innovations in Plant Breeding,” Nature Plants 6:610-9 (2020)), it is highly genotype dependent. In vivo haploid production by inter- or intra-specific hybridization is mainly limited to monocots. Also, manipulation of the centromeric histone protein CENH3 and other known genes involved in gamete incompatibility (Msi2, DMP8, and ZmPOD65) have not yet been extensively applied to many important crop species, especially dicots. A genotype-independent and stable haploid induction system, therefore, is still required for rapid development of homozygous (inbred) lines used in most crop breeding, including Cannabis.

Identification of the genetic loci in the stock 6 QTL involved with haploid induction: Important information has been discovered by independent researchers to reveal the genetics of haploid induction, which was first reported by Sprague in 1929. A causative locus for heritable genetic haploid induction was first found in stock 6 maize, by Coe, 1959. The causative genetic element in stock 6 maize was mapped to the QTL complexes qhir 1 and qhir 8 (Zhong et al., “A DMP-triggered in vivo maternal haploid induction system in the dicotyledonous Arabidopsis,” Nature Plants 6:466-72 (2020)). QTL qhir 1 was found to be caused by a four-base pair insertion at the 3′ of the coding region of a gene given different names by the different researchers. This locus is now referred to as ZmPLA1/MTL/NLD (patatin-like phospholipaseA1/MATRILINEAL/NOT LIKE DAD), which encodes a sperm cell-expressed phospholipase A (Jacquier et al., “Puzzling Out Plant Reproduction by Haploid Induction for Innovations in Plant Breeding,” Nature Plants 6:610-9 (2020)). The causative allele at this locus has been used extensively in monocot crops for F1 hybrid production and other purposes (Hu et al., “The genetic basis of haploid induction in maize identified with a novel genome-wide association method,” Genetics 202(4):1267-76 (2016)). The causative allele for haploid induction in the qhir8 QTL is a single-nucleotide change in the pollen-expressed gene DMP (DOMAIN OF UNKNOWN FUNCTION 679 membrane protein), which leads to a single amino acid substitution in the first predicted transmembrane domain 4 (Zhong et al, “A DMP-triggered in vivo maternal haploid induction system in the dicotyledonous Arabidopsis,” Nature Plants 6:466-72 (2020); Zhong et al., “In vivo material haploid induction in tomato,” Plant Biotechnol. J. 20(2):250-2 (2022)). The Haploid Induction Rates (HIRs) in ZmPLA1 and ZmDMP mutants are 2-3%, which can be increased to 6-10% when the zmpla1 and zmdmp alleles are combined. Loss of function of ZmPLA1 orthologues in rice, wheat, and other species also trigger maternal haploid induction, but this approach has not been extensively applied to dicots, owing to the lack of identifiable ZmPLA1 ortholog genes in the numerous dicot crops.

General use of the DMP8 haploid induction gene: The recent report of the conservation of the DMP8 gene sequences in dicot species has opened the possibility to apply this haploid induction system in many crops in general, especially dicots (Zhong et al., “A DMP-triggered in vivo maternal haploid induction system in the dicotyledonous Arabidopsis,” Nature Plants 6:466-72 (2020)). However, although DMP8 loss-of-function mutants have been used to obtain haploid lines in corn (Jacquier et al., “Maize in Planta Haploid Inducer Lines: A Cornerstone for Doubled Haploid Technology,” Doubled Haploid Technology 2288:25-48 (2021); Meng et al., “Haploid induction and its application in maize breeding,” Mol. Breeding 41:3 (2021)), Medicago (Wang et al., “In planta haploid induction by genome editing of DMP in the model legume Medicago truncatula,” Plant Biotechnol. J. 20(1):22-24 (2022)), tomato (Zhong et al., “A DMP-triggered in vivo maternal haploid induction system in the dicotyledonous Arabidopsis,” Nature Plants 6:466-72 (2020)), rice (Yao et al., “OsMATL mutation induces haploid seed formation in indica rise,” Nat. Plants. 4:530-3 (2018)), and wheat (Liu et al., “Extension of the in vivo haploid induction system from diploid maize to hexaploidy wheat,” Plant Biotechnol. J. 18(4316-8 (2020)), and nicotiana through CRISPR Technology, CRISPR mediated mutations that are inherited through seeds are still not possible with Cannabis. Although there are reports of stable CRISPR, edits of Cannabis, these are not transferred through germ lines (Zhang et al., “Establishment of an Agrobacterium-mediated genetic transformation and CRISPR/Cas9-mediated targeted mutagenesis in hemp (Cannabis sativa. L.),” Plant Biotechnol. J. doi.org/10.1111/pbi.13611 (2021)).

Therefore, as described herein, the discovery of a novel naturally occurring misfunction mutation in the sperm expressed DMP8 allele of Cannabis, allows the creation of a useful haploid inducer phenotype, and subsequent haploid/dihaploids in any taxa of Cannabis, identified through screening for DMP8 mutations or transfer by crossing any taxa to DMP8 Inducer C, or to Inducer K lines. Haploid segregates are identified by a combination of phenotype markers and DNA sequencing to determine the absence or presence of SNP markers.

### BRIEF SUMMARY OF THE INVENTION

Provided herein are methods of producing a Cannabis haploid/di-haploid plant in a single generation. The methods comprise (a) obtaining pollen comprising a mutation in DMP8; (b) using the pollen to pollinate a Cannabis plant; (c) collecting a seed from the pollenated Cannabis plant; (d) identifying putative haploid Cannabis plants from the seed; and (e) producing a Cannabis haploid/di-haploid plant in a single generation.

In certain embodiments, the mutation in DMP8 results in a deletion of a single amino acid in the amino terminus of DMP8. In certain embodiments, the mutation in DMP8 is encoded by a nucleic acid sequence comprising at least 80% identity with SEQ ID NO:4 or SEQ ID NO:5. In certain embodiments, the mutation in DMP8 is encoded by the nucleic acid sequence of SEQ ID NO: 4 or SEQ ID NO: 5.

In certain embodiments, the Cannabis plant is selected from the group consisting of Cannabis sativa, Cannabis indica, and Cannabis ruderalis.

In certain embodiments, the Cannabis haploid/di-haploid plant produced is found by a phenotypic indicator. The phenotypic indicator can, for example, be selected from the group consisting of dwarfism, ectopic hair/trichome presence on leaves, and a different leaf structure. The different leaf structure can, for example, comprise leaves with 3 leaflets with 7 or 8 lobes in the central and lateral leaflets.

In certain embodiments, the methods further comprise the step of confirming the putative haploid Cannabis plant utilizing genetic techniques. The genetic technique can, for example, be selected from chromosome counting, seed sterility, and single nucleotide polymorphism (SNP) genetic analysis. In certain embodiments, chromosome counting is performed by flow cytometry. In certain embodiments, the SNP genetic analysis is confirmed via DNA sequencing.

In certain embodiments, the produced Cannabis haploid/di-haploid plant does not comprise the mutation in DMP8.

In certain embodiments, the methods further comprise (f) obtaining a seed from the produced Cannabis haploid/di-haploid plant. In certain embodiments, the methods further comprise (g) growing a Cannabis haploid/di-haploid plant from the seed.

Also provided are Cannabis haploid/di-haploid plants produced by the methods as described herein. In certain embodiments, the Cannabis haploid/di-haploid plant comprises cells, tissues, and organs. The organs can, for example, include seeds, leaves, fruits, flowers, stems, and/or roots. In certain embodiments, the Cannabis haploid/di-haploid plant includes propagation materials selected from the group consisting of pollen, ovaries, ovules, germs, endosperms, egg cells, cleavage, roots, root tips, hypotcotyls, cotyledons, stems, leaves, flowers, anthers, seeds, meristematic cells, protoplasts, and tissue cultures.

Also provided is a Cannabis haploid/di-haploid seed obtained by the methods described herein. The Cannabis haploid/di-haploid seed can, for example, be genetically identical or nearly identical with the pollenated Cannabis plant.

### BRIEF DESCRIPTION OF DRAWINGS

The foregoing summary, as well as the following detailed description of the preferred embodiments of the present application, will be better understood when read in conjunction with the appended drawings. It should be understood, however, that the application is not limited to the precise embodiments shown in the drawings.

FIG. 1 shows a nucleotide sequence alignment of DMP8 coding sequence obtained from Cannabis accessions (Sativa, Ruderalis) and mutants (Inducer K, Inducer C) to a reference DMP8 sequence. Shaded box highlights the sequence mutation underlying haploid inducer phenotype. Sequences used to obtain the alignment are given at the end of the examples section.

FIG. 2 shows an image of dwarfism in putative Cannabis spp. Haploids (left) compared to diploid individuals (right) resulting from pollination with haploid inducer lines.

FIG. 3 shows images of ectopic presence of hair/trichomes in putative Cannabis spp. haploids (FIG. 3A) compared to diploid individuals (FIG. 3B) resulting from pollination with haploid inducer lines.

FIG. 4 shows an image of the modified leaf structure in putative Cannabis spp. haploids (right) compared to diploid individuals (left) resulting from pollination with haploid inducer lines.

FIG. 5 shows the flow cytometry analysis in putative Cannabis spp. haploids (P3) compared to diploid individuals (P2) resulting from pollination with haploid inducer lines.

FIG. 6 shows an image of seed morphology of putative Cannabis spp. haploids (FIG. 6A) compared to diploid individuals (FIG. 6B) resulting from pollination with haploid inducer lines.

FIGS. 7A and 7B show images of the sequencing of putative haploids and diploids in the SNP-1 containing region (FIG. 7A) and the sequencing of the SNPs of putative haploid 4 (PH4) (FIG. 7B).

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

The words “right,” “left,” “lower,” and “upper” designate directions in the drawings to which reference is made.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., nucleic acid sequences encoding DMP8 and mutations thereof), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 1981; 2:482, by the homology alignment algorithm of Needleman & Wunsch, J. Mol. Biol. 1970; 48:443, by the search for similarity method of Pearson & Lipman, Proc. Nat'l. Acad. Sci. USA 1988; 85:2444, by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., 1995 Supplement (Ausubel)).

Examples of algorithms that are suitable for determining percent sequence identity and sequence similarity are the BLAST and BLAST 2.0 algorithms, which are described in Altschul et al., J. Mol. Biol. 1990; 215: 403-410 and Altschul et al., Nucleic Acids Res. 1997; 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 1989; 89:10915).

In addition to calculating percent sequence identity, the BLAST algorithm also performs a statistical analysis of the similarity between two sequences (see, e.g., Karlin & Altschul, Proc. Nat'l. Acad. Sci. USA 1993; 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein, the term “gene” refers to a hereditary unit including a sequence of DNA that occupies a specific location on a chromosome and that contains the genetic instructions for a particular characteristic or trait in an organism.

As used herein, a plant referred to as “haploid” has a single set (genome) of chromosomes and the reduced number of chromosomes (n) in the haploid plant is equal to that of the gamete.

As used herein, the term “phenotypic indicator” refers to one or more traits of a plant or a plant cell. The phenotype can be observable to the naked eye, or by any means of evaluation known in the art, e.g., microscopy or biochemical analysis. In certain cases, a phenotype is directly controlled by a single gene or a genetic locus.

As used herein, the term “plant” can refer to the whole plant, any part thereof, or a cell or tissue culture derived from the plant.

A plant cell is a cell of a plant, taken from a plant, or derived through culture from a cell taken from the plant. Thus, the term “plant cell” includes, without limitation, cells within seeds, suspension cultures, embryos, meristematic regions, callus tissue, leaves, shoots, gametophytes, sporophytes, pollen, and microspores. The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds.

The invention consists of a method using specific Haploid Inducer lines to produce Cannabis haploid plants and consequent spontaneous or chemically induced haploids/di-haploids in one generation. Naturally occurring haploid inducer lines can be used to generate completely homozygous di-haploid lines from any heterozygous Cannabis plants. This technology provides a genetic platform to accomplish several breeding goals in one or two generations, that presently require up to 12 generations of selfing or backcrossing. The di-haploids obliviate the need to use multi-generational crosses to produce inbred or homozygous lines. These include but are not limited to the ability to combine or enhance phenotypic traits and establish uniformity and heterosis in true F1 hybrids. This platform can also be used to very rapidly create large numbers of homozygous recombinant (inbreds) lines for QTL (quantitative trait loci) or GWAS (genome wide association study) analyses (Hu et al., “The genetic basis of haploid induction in maize identified with a novel genome-wide association method,” Genetics 202(4):1267-76 (2016)), and other goals of reverse genetics or reverse breeding, such as the identification of parental gamete genomes present in important, useful, and valuable heterozygous lines. The combination of these identified gametes (haploid/di-haploid plants) by crossing may be used for recreation and clonal multiplication of such heterozygous lines. These valuable vegetatively clonally multiplied lines, or their selfed (Si) progeny and only the genome sequences of the parents of these lines obtained by genome sequencing of non-living whole tissue (e.g., purchased at a retail location) can be used to “recreate” the living parental lines for any practical use. The parents can be “brought back to life” for any use of interest. Crossing such “recreated” parents will produce only progeny genetically identical to the heterozygous progeny used as the commercial product being sold at retailers and elsewhere. This technology also may be used for the identification of haplotypes in heterozygous lines, to rapidly fix genetic information once an interesting gene combination is obtained (Srivarsha et al., “Reverse Breeding-A Breakthrough Approach of Modern Plant Breeding,” Int. J. Biotechnol. Biomed. Sci. 4(2):74-78 (2018)).

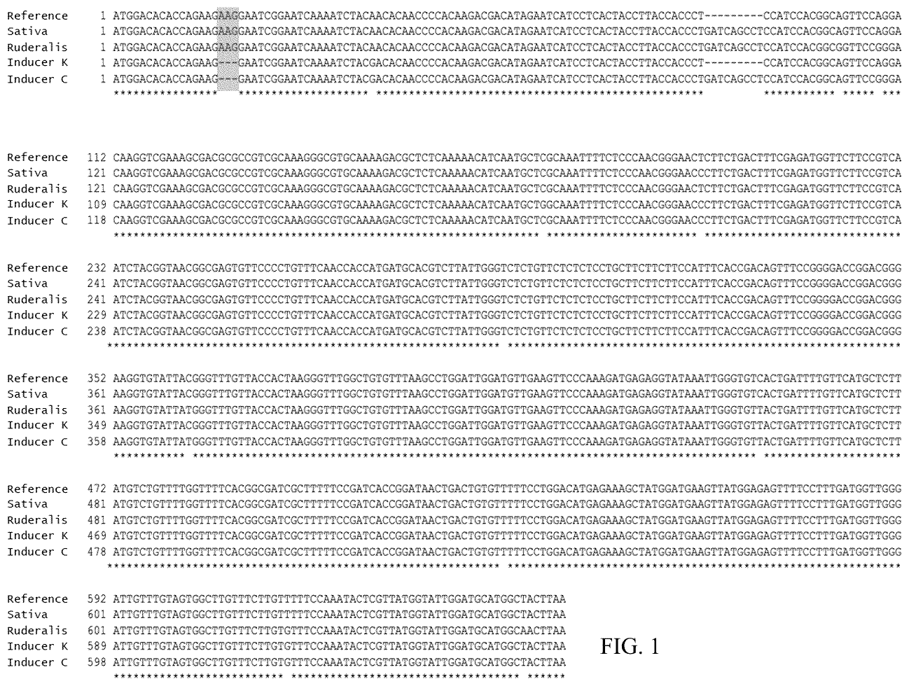

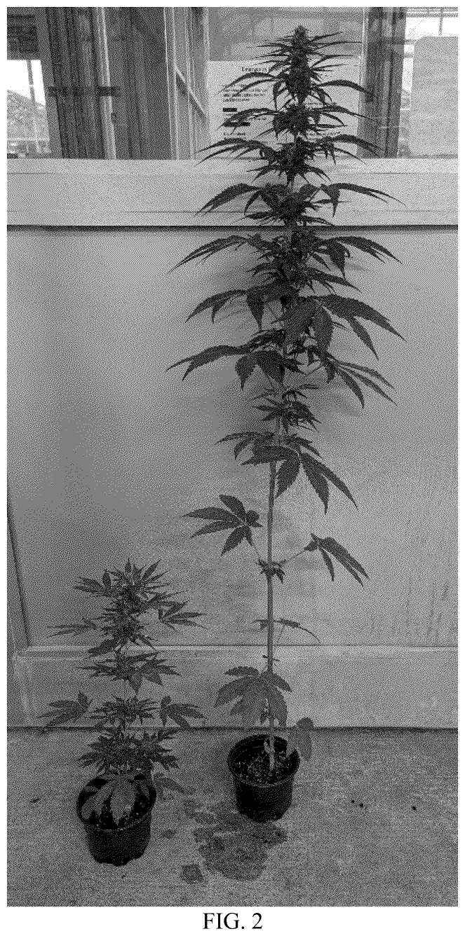

It is disclosed herein that pollination of any Cannabis genotype/phenotype named by any nomenclature, used to denote and describe various lineages of the originally named species Cannabis sativa L., with pollen from naturally occurring haploid inducers (“Inducer K, Inducer C”, FIG. 1), containing naturally occurring or physically or chemically induced misfunctional mutations at the DMP8 locus (Zhong et al., “A DMP-triggered in vivo maternal haploid induction system in the dicotyledonous Arabidopsis,” Nature Plants 6:466-72 (2020)), results in haploid induction. The Cannabis haploid inducers do not have clear and consistent morphological distinctive traits but can be identified by sequence analysis of the DMP8 gene portion encoding the protein N terminus (here generically called “marker”), where a single amino acid deletion results in a misfunctional DMP8.

Thus, provided herein are methods of producing a Cannabis haploid/di-haploid plant in a single generation. The methods comprise (a) obtaining pollen comprising a mutation in DMP8; (b) using the pollen to pollinate a Cannabis plant; (c) collecting a seed from the pollenated Cannabis plant; (d) identifying putative haploid Cannabis plants from the seed; and (e) producing a Cannabis haploid/di-haploid plant in a single generation.

In certain embodiments, the mutation in DMP8 results in a deletion of a single amino acid in the amino terminus of DMP8. In certain embodiments, the mutation in DMP8 is encoded by a nucleic acid sequence comprising at least 80% identity with SEQ ID NO:4 or SEQ ID NO:5. The nucleic acid sequence can, for example, comprise at least 80%, at least 85%, at least 90%, at least 95%, or at least 99% identity with SEQ ID NO:4 or SEQ ID NO:5. The nucleic acid sequence can, for example, be 80%, 81%, 82%, 83%, 84%, 85%, 86%, 87%, 88%, 89%, 90%, 91%, 92%, 93%, 94%, 95%, 96%, 97%, 98%, 99%, or 100% identical with SEQ ID NO:4 or SEQ ID NO:5. In certain embodiments, the mutation in DMP8 is encoded by the nucleic acid sequence of SEQ ID NO: 4 or SEQ ID NO: 5.

In certain embodiments, the Cannabis plant is selected from the group consisting of Cannabis sativa, Cannabis indica, and Cannabis ruderalis.

In certain embodiments, the Cannabis haploid/di-haploid plant produced is identified by a phenotypic indicator. The phenotypic indicator can, for example, be selected from the group consisting of dwarfism, ectopic hair/trichome presence on leaves, and a different leaf structure. The different leaf structure can, for example, comprise leaves with three (3) leaflets with seven (7) or eight (8) lobes in the central and lateral leaflets.

In certain embodiments, the methods further comprise the step of confirming the putative haploid Cannabis plant utilizing genetic techniques. The genetic technique can, for example, be selected from chromosome counting, seed sterility, and single nucleotide polymorphism (SNP) genetic analyses. In certain embodiments, chromosome counting is performed by flow cytometry. In certain embodiments, the SNP genetic analysis is confirmed via DNA sequencing.

In certain embodiments, the produced Cannabis haploid/di-haploid plant does not comprise the mutation in DMP8, but is capable of inducing haploid plants from elite genetic material.

In certain embodiments, the methods further comprise (f) obtaining a seed from the produced Cannabis haploid/di-haploid plant. In certain embodiments, the methods further comprise (g) growing a Cannabis haploid/di-haploid plant from the seed.

Also provided are Cannabis haploid/di-haploid plants produced by the methods as described herein. In certain embodiments, the Cannabis haploid/di-haploid plant comprises cells, tissues, and organs. The organs can, for example, include seeds, leaves, fruits, flowers, stems, and/or roots. In certain embodiments, the Cannabis haploid/di-haploid plant includes propagation materials selected from the group consisting of pollen, ovaries, ovules, germs, endosperms, egg cells, cleavage, roots, root tips, hypotcotyls, cotyledons, stems, leaves, flowers, anthers, seeds, meristematic cells, protoplasts, and tissue cultures.

Also provided is a Cannabis haploid/di-haploid seed obtained by the methods described herein. The Cannabis haploid/di-haploid seed can, for example, be genetically identical with the pollenated Cannabis plant.

### EMBODIMENTS

The invention provides the following non-limiting embodiments.

Embodiment 1 is a method of producing a Cannabis haploid/di-haploid plant in a single generation, the method comprising:
(a) obtaining pollen comprising a mutation in DMP8;
(b) using the pollen to pollinate a Cannabis plant;
(c) collecting a seed from the pollenated Cannabis plant
(d) identifying putative haploid Cannabis plants from the seed; and
(e) producing a Cannabis haploid/di-haploid plant in a single generation.

Embodiment 2 is the method of embodiment 1, wherein the mutation in DMP8 results in a deletion of a single amino acid in the amino terminus of DMP8.

Embodiment 2a is the method of embodiment 2, wherein the mutation in DMP8 is encoded by a nucleic acid sequence comprising at least 80% identity to SEQ ID NO: 4 or SEQ ID NO: 5.

Embodiment 3 is the method of embodiment 2 or 2a, wherein the mutation in DMP8 is encoded by the nucleic acid sequence of SEQ ID NO:4 or SEQ ID NO:5.

Embodiment 4 is the method of any one of embodiments 1-3, wherein the Cannabis plant is selected from the group consisting of Cannabis sativa, Cannabis indica, and Cannabis ruderalis.

Embodiment 5 is the method of any one of embodiments 1-4, wherein the Cannabis haploid/di-haploid plant produced is identified by a phenotypic indicator.

Embodiment 6 is the method of embodiment 5, wherein the phenotypic indicator is selected from the group consisting of dwarfism, ectopic hair/trichome presence on leaves, and a different leaf structure.

Embodiment 7 is the method of embodiment 6, wherein the different leaf structure comprises leaves with 3 leaflets with 7 or 8 lobes in the central and lateral leaflets.

Embodiment 8 is the method of any one of embodiments 1-7, further comprising the step of confirming the putative haploid Cannabis plant utilizing genetic techniques.

Embodiment 9 is the method of embodiment 8, wherein the genetic technique is selected from chromosome counting, seed sterility, and single nucleotide polymorphism (SNP) genetic analysis.

Embodiment 10 is the method of embodiment 9, wherein the chromosome counting is performed by flow cytometry.

Embodiment 11 is the method of embodiment 9, wherein the SNP genetic analysis is confirmed via DNA sequencing.

Embodiment 12 is the method of any one of embodiments 1-11, wherein the Cannabis haploid/di-haploid plant does not comprise the mutation in DMP8.

Embodiment 13 is a Cannabis haploid/di-haploid plant produced by the method of any one of embodiments 1-12.

Embodiment 14 is the Cannabis haploid/di-haploid plant of embodiment 13, where the Cannabis haploid/di-haploid plant comprises cells, tissues, and organs.

Embodiment 15 is the Cannabis haploid/di-haploid plant of embodiment 14, wherein the organs include seeds, leaves, fruits, flowers, stems, and/or roots.

Embodiment 16 is the Cannabis haploid/di-haploid plant of any one of embodiments 13-15, wherein the Cannabis haploid/di-haploid plant includes propagation materials selected from the group consisting of pollen, ovaries, ovules, germs, endosperms, egg cells, cleavage, roots, root tips, hypotcotyls, cotyledons, stems, leaves, flowers, anthers, seeds, meristematic cells, protoplasts, and tissue cultures.

Embodiment 17 is the method of any one of embodiments 1-12, further comprising:
(f) obtaining a seed from the produced Cannabis haploid/di-haploid plant.

Embodiment 18 is the method of embodiment 13, further comprising:
(g) growing a Cannabis haploid/di-haploid plant from the seed.

Embodiment 19 is a Cannabis haploid/di-haploid seed obtained by the method of embodiment 17.

Embodiment 20 is the Cannabis haploid/di-haploid seed of embodiment 19, wherein the seed is genetically identical with the pollenated Cannabis plant.

### EXAMPLES

The sequence analysis of the DMP8 gene reveals mutation(s) at the 5′ end of the coding sequence (here called “genetic marker”) and identifies a haploid-inducer phenotype. The DMP8-like Coding DNA sequence (CDS) alignment (FIG. 1) illustrates this genetic marker. This marker is one method to define and identify the specific male parent genotypes capable of haploid induction in a subset of the resulting progeny, when used for pollination of female flowers of any Cannabis taxa. Specifically, the mutation carried by inducer genotypes “Inducer C” and “Inducer K” at the 5′ end of the DMP8 locus is responsible for a one amino acid deletion in the encoded protein (FIG. 1). The male haploid inducer gametes from these plants, that carry this deletion, do not transfer any coding DNA to the female gamete when crossed. The unfertilized egg follows a parthenogenesis biological pathway, creating a maternal haploid embryo. Therefore, the DMP8 mutation is not transferred to the haploids (Zhong et al., “A DMP-triggered in vivo maternal haploid induction system in the dicotyledonous Arabidopsis,” Nature Plants 6:466-72 (2020)).

### Haploidy Confirmation

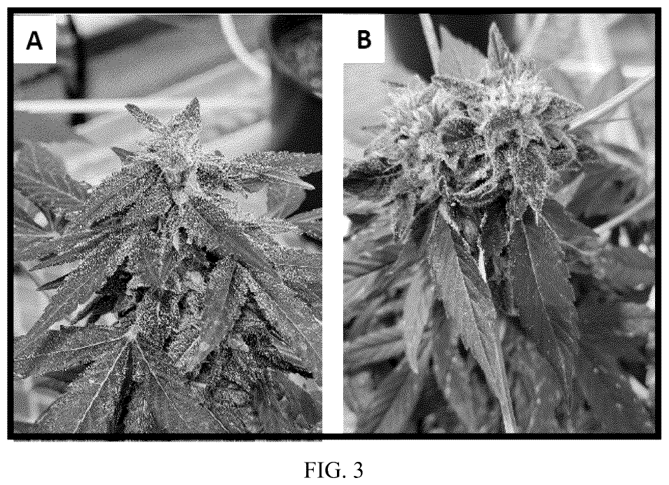

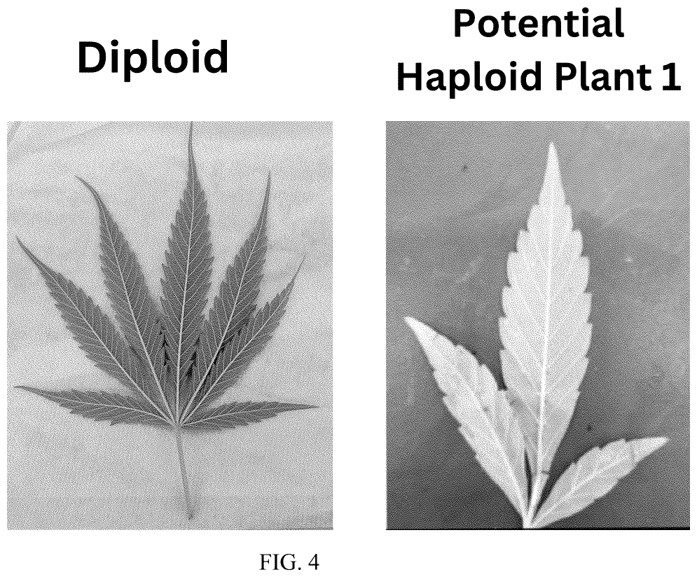

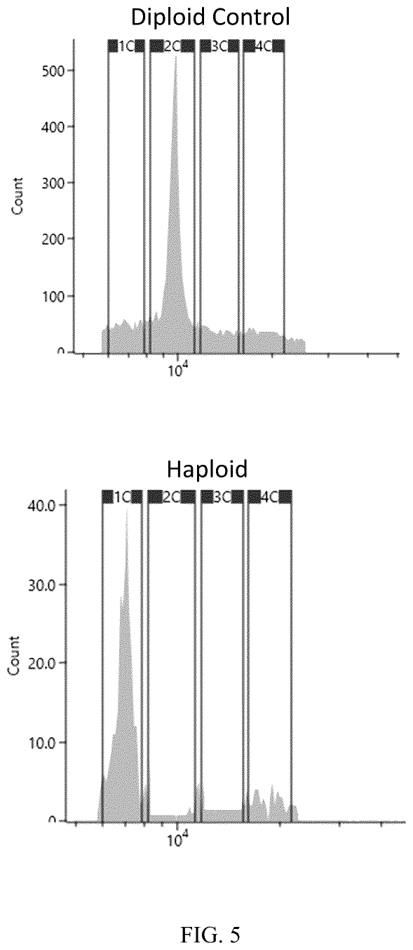

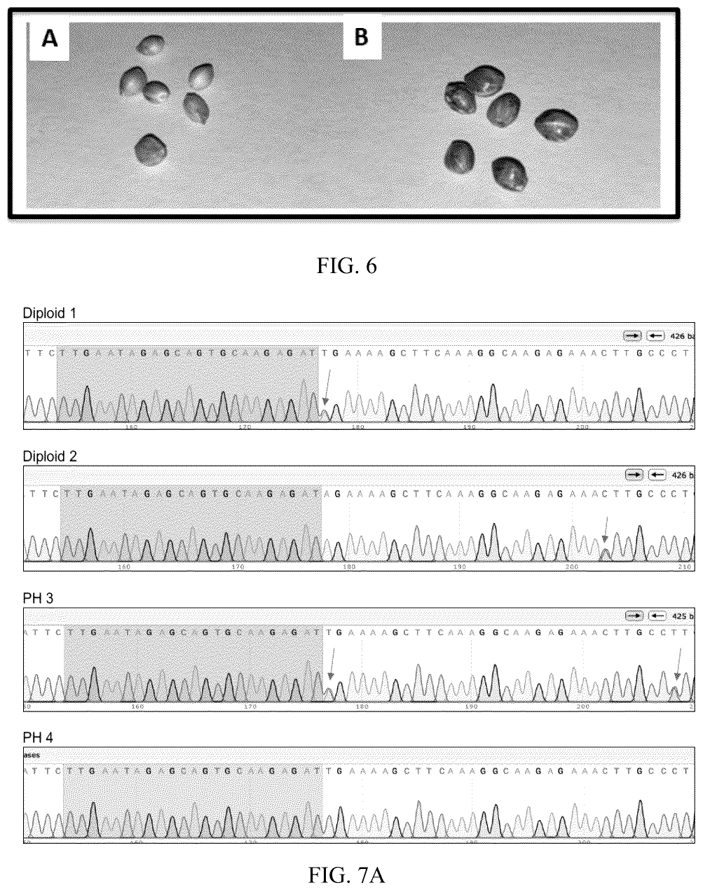

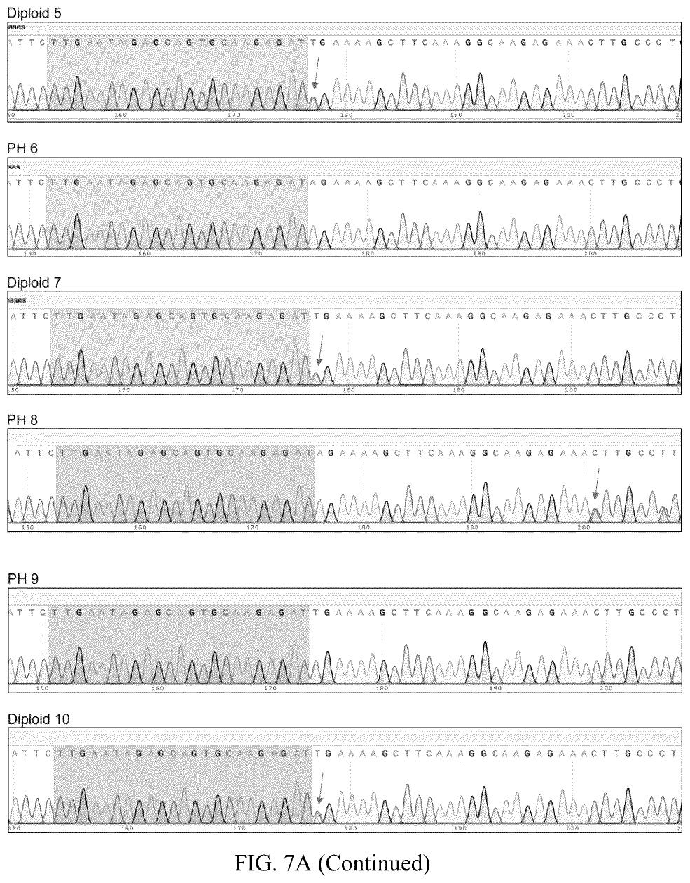

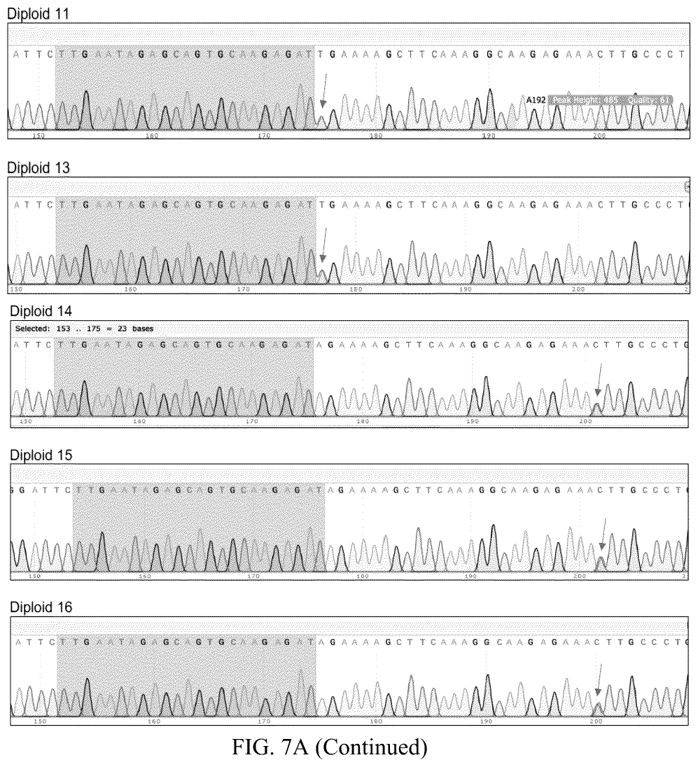

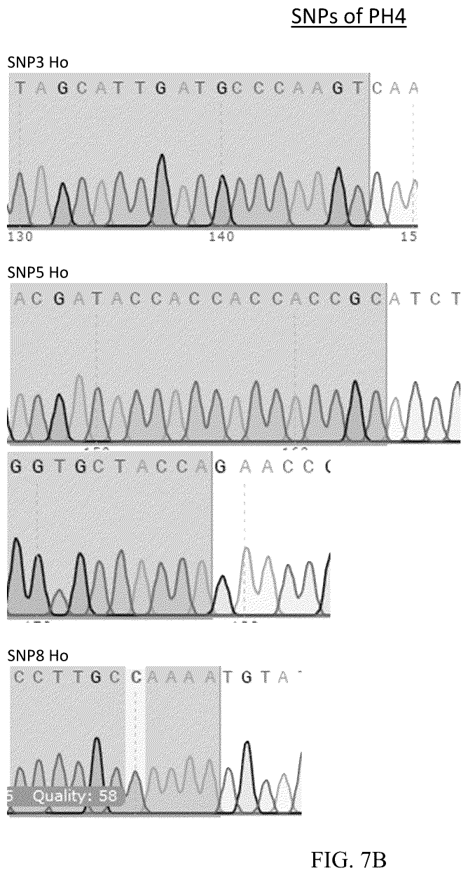

Because haploidy is induced in a subset of the individuals obtained after pollination with haploid inducer lines, haploids need to be identified in the progeny through some phenotypic indicators of haploidy (useful for large scale screening) and confirmed through genetic analyses. Indicators of haploidy include dwarfism (FIG. 2), ectopic hair/trichome presence on leaves (FIG. 3), and a different leaf structure (FIG. 4). In detail, structure changes suggestive of haploidy, sometimes present on some haploids, were leaves composed of 3 leaflets, with 8 or 7 lobes in the central and lateral leaflets, respectively. Whereas leaves from diploids normally have 5 leaflets with 14 lobes in the central leaflet and 8 lobes for the lateral leaflets. Genetic proof is given by chromosome staining, and flow cytometry/chromosome counts, showing a lower chromosome count (never more than 10) in haploids (FIGS. 5A-5B). Also, seed sterility (FIG. 6) and lack of SNP variation at known haplotype regions of the wild type plant genomes (Table 1) were definitive haploid traits.

TABLE 1
From 60 progeny of a male inducer C × female Cannabis plant, 16 putative haploid plants were chosen that displayed at least one of the
haploid phenotypes. Eight SNP-containing loci on each of the different chromosomes of these plants were identified from the parents of the
haploid induction cross and they were all checked for homozygosity or heterozygosity by deep sequencing. One of these putative haploids was
homozygous (PH4) at three SNP loci (SNP 1, 2, 8). All ten diploid control plants were heterozygous at all of the SNP loci checked by sequencing.

| | | | | | | | | | | | | | | | | | Inducer K | Inducer C |
|---|---|---|---|---|---|---|---|---|---|---|---|---|---|---|---|---|---|---|
| | PH1 | PH2 | PH3 | PH4 | PH5 | PH6 | PH7 | PH8 | PH9 | PH10 | PH11 | PH12 | PH13 | PH14 | PH15 | PH16 | (Diploid) | (Diploid) |
| SNP1 | He | He | He | Ho | He | Ho | He | He | Ho | He | He | n.a | He | He | He | He | Ho | He |
| | 177 | 204 | 178 | | 177 | | 177 | 201 | | 177 | 175 | | 177 | 231 | 202 | 201 | | |
| | | | 288 | | | | | | | | | | | | | | | |
| SNP2 | Ho | He | Ho | Ho | He | Ho | He | Ho | Ho | He | He | Ho | He | He | He | Ho | He | He |
| SNP3 | | | | Ho | | | | | | | | | | | | | | |
| SNP4 | | | | Ho | | | | | | | | | | | | | | |
| SNP5 | | | | Ho | | | | | | | | | | | | | | |
| SNP8 | | | | Ho | | | | | | | | | | | | | | |
| SNP9 | | | | Ho | | | | | | | | | | | | | | |
| DMP8 | | | | Ho | | n.a | | | Ho | | | | | | | | Ho | |

PH: Putative haploid;
He: heterozygous, with SNP;
Ho: homozygous, without SNP;
n.a.: sequencing failed

Indeed, because haploids only carry one allele of each locus, lack of heterozygosity in polymorphic sequences located on different chromosomes can be used as highly probable evidence of haploidy. In the example reported in Table 1, these putative haploids first identified by plant morphology (PH1-16), presenting at least one of the haploid indicator morphology phenotypes, were selected for determination of allele sequence variation in genome sequence haplotypes known from Cannabis genome data based to be widely present among diverse Cannabis lineages (Welling et al., “An Extreme-Phenotype Genome-Wide Association Study Identifies Candidate Cannabinoid Pathway Genes in Cannabis,” Scientific Reports Vol. 10, Article 18643 (2020)). Selection of the specific SNPs used to confirm haploidy was based on their presence in genetic backgrounds of both the haploid inducer and the female parents used in our experiments to generate haploids. As shown in table 1, (PH4) individuals presented no sequence variants at any of the haplotypes identified, indicating strong evidence for haploidy in these individuals. In fact, the probability of homozygosity by independent assortment of gametes in this cross is 1:250 individual progeny. However, it was found that haploid plants occurred at a frequency of 1:16. This was confirmed by chromosome counting with flow cytometry (FIG. 5). All the diploid controls had at least one SNP that identified lineage specificity at the chosen haplotype genome regions.

### REFERENCES

1. Hu, H et al., 2016 The Genetic Basis of Haploid Induction in Maize Identified with a Novel Genome-Wide Association Method. Genetics Society of America, DOI
2. Jacquier, N et al., 2021. Maize In Planta Haploid Inducer Lines: A Cornerstone for Doubled Haploid Technology. Doubled Haploid Technology, 2288, pp. 25-48.
3. Jacquier, N et al., 2020. Puzzling Out Plant Reproduction by Haploid Induction for Innovations in Plant Breeding. Nature Plants, Vol 6, June 2020, 610-619
4. Jiang, C et al., 2022. ROS Triggers Haploid induction. Molecular Plant, doi.org/10.1016/j.molp.2022.04.001
5. Kurtz, L et al, 2020. Production of Tetraploid and Triploid Hemp. Hort Science, Vol. 1703-1707
6. Liu C et al., 2020. Extension of the in vivo haploid induction system from diploid maize to hexaploid wheat. Plant biotechnology journal, 18(2), 316-318. doi.org/10.1111/pbi.13218.
7. McMahon, K, 2017. Double Haploid Induction Speeds Up Plant Breeding Process. Syngenta Thrive, Vol. Summer 2017
8. Meng, D et al., 2021. Haploid induction and its application in maize breeding. Mol Breeding 41, 20. doi.org/10.1007/s11032-021-01204-5
9. Parsons, J et al., 2019. Polyploidization fix the Genetic Improvement of Cannabis sativa. Frontiers in Plant Science, doi.org/10.3389/fpls.2019.00476
10. Srivarsha, J et al., 2018. Reverse Breeding-A Breakthrough Approach of Modern Plant Breeding. International Journal of Biotechnology and Biomedical Sciences Vol 4. issue 2 pp. 74-78
11. Wang, Na et al., 2022. In planta haploid induction by genome editing of DMP in the model legume Medicago truncatula. Plant biotechnology journal vol. 20, 1: 22-24. doi:10.1111/pbi.13740.
12. Welling, M et al., 2020. An Extreme-Phenotype Genome-Wide Association Study Identifies Candidate Cannabinoid Pathway Genes in Cannabis. Scientific Reports Vol 10, Article Number: 18643
13. Yao L et al., 2018. OsMATL mutation induces haploid seed formation in indica rice. Nat Plants, 4, 530-533.
14. Zhang, X et al., 2021. Establishment of an Agrobacterium-Mediated Genetic Transformation and CRISPR/Cas9-Mediated Targeted Mutagenesis in Hemp (Cannabis Sativa L.) Plant Biotechnology Journal (2021), doi.org/10.1111/pbi.13611
15. Zhong, Y et al., 2020. A DMP-Triggered In Vivo Maternal Haploid Induction System in the Dicotyledonous Arabidopsis. Nature Plants. 6.466-472.
16. Zhong Y et al., 2022. In Vivo Maternal Haploid Induction in Tomato. Plant Biotechnol J. 20(2):250-252. doi: 10.1111/pbi.13755.

### PATENTS

CNA 136317115A—“Novel wheat CENH3 allele”.
WO2017087682A1—“Haploid Induction Compositions and Methods for Use Therefor”
WO2020239137—“Parthenogenic Haploid Induction Gene DMP and Application Thereof”
US202100765871—“Method for the Production of Haploid and Subsequent Doubled Haploid Plants”
CN104046694B—“A Kind Ofly to Identify or Whether Assistant Identification Arabidopsis thaliana to be Measured is the Method for Male Sterile Strain

SEQUENCES
DMP8-like CDS Sequence (Reference)
SEQ ID NO: 1
ATGGACACACCAGAAGAAGGAATCGGAATCAAAATCTACA
ACACAACCCCACAAGACGACATAGAATCATCCTCACTACC
TTACCACCCTCCATCCACGGCAGTTCCAGGACAAGGTCGA
AAGCGACGCGCCGTCGCAAAGGGCGTGCAAAAGACGCTCT
CAAAAACATCAATGCTCGCAAATTTTCTCCCAACGGGAAC
TCTTCTGACTTTCGAGATGGTTCTTCCGTCAATCTACGGT
AACGGCGAGTGTTCCCCTGTTTCAACCACCATGATGCACG
TCTTATTGGGTCTCTGTTCTCTCTCCTGCTTCTTCTTCCA
TTTCACCGACAGTTTCCGGGGACCGGACGGGAAGGTGTAT
TACGGGTTTGTTACCACTAAGGGTTTGGCTGTGTTTAAGC
CTGGATTGGATGTTGAAGTTCCCAAAGATGAGAGGTATAA
ATTGGGTGTCACTGATTTTGTTCATGCTCTTATGTCTGTT
TTGGTTTTCACGGCGATCGCTTTTTCCGATCACCGGATAA
CTGACTGTGTTTTTCCTGGACATGAGAAAGCTATGGATGA
AGTTATGGAGAGTTTTCCTTTGATGGTTGGGATTGTTTGT
AGTGGCTTGTTTCTTGTTTTTCCAAATACTCGTTATGGTA
TTGGATGCATGGCTACTTAA
DMP8-like CDS Sequence (Sativa)
SEQ ID NO: 2
ATGGACACACCAGAAGAAGGAATCGGAATCAAAATCTACA
ACACAACCCCACAAGACGACATAGAATCATCCTCACTACC
TTACCACCCTGATCAGCCTCCATCCACGGCAGTTCCAGGA
CAAGGTCGAAAGCGACGCGCCGTCGCAAAGGGCGTGCAAA
AGACGCTCTCAAAAACATCAATGCTCGCAAATTTTCTCCC
AACGGGAACCCTTCTGACTTTCGAGATGGTTCTTCCGTCA
ATCTACGGTAACGGCGAGTGTTCCCCTGTTTCAACCACCA
TGATGCACGTCTTATTGGGTCTCTGTTCTCTCTCCTGCTT
CTTCTTCCATTTCACCGACAGTTTCCGGGGACCGGACGGG
AAGGTGTATTACGGGTTTGTTACCACTAAGGGTTTGGCTG
TGTTTAAGCCTGGATTGGATGTTGAAGTTCCCAAAGATGA
GAGGTATAAATTGGGTGTCACTGATTTTGTTCATGCTCTT
ATGTCTGTTTTGGTTTTCACGGCGATCGCTTTTTCCGATC
ACCGGATAACTGACTGTGTTTTTCCTGGACATGAGAAAGC
TATGGATGAAGTTATGGAGAGTTTTCCTTTGATGGTTGGG
ATTGTTTGTAGTGGCTTGTTTCTTGTTTTTCCAAATACTC
GTTATGGTATTGGATGCATGGCTACTTAA
DMP8-like CDS Sequence (Ruderalis)
SEQ ID NO: 3
ATGGACACACCAGAAGAAGGAATCGGAATCAAAATCTACA
ACACAACCCCACAAGACGACATAGAATCATCCTCACTACC
TTACCACCCTGATCAGCCTCCATCCACGGCGGTTCCGGGA
CAAGGTCGAAAGCGACGCGCCGTCGCAAAGGGCGTGCAAA
AGACGCTCTCAAAAACATCAATGCTCGCAAATTTTCTCCC
AACGGGAACTCTTCTGACTTTCGAGATGGTTCTTCCGTCA
ATCTACGGTAACGGCGAGTGTTCCCCTGTTTCAACCACCA
TGATGCACGTCTTATTGGGTCTCTGTTCTCTCTCCTGCTT
CTTCTTCCATTTCACCGACAGTTTCCGGGGACCGGACGGG
AAGGTGTATTATGGGTTTGTTACCACTAAGGGTTTGGCTG
TGTTTAAGCCTGGATTGGATGTTGAAGTTCCCAAAGATGA
GAGGTATAAATTGGGTGTTACTGATTTTGTTCATGCTCTT
ATGTCTGTTTTGGTTTTCACGGCGATCGCTTTTTCCGATC
ACCGGATAACTGACTGTGTTTTTCCTGGACATGAGAAAGC
TATGGATGAAGTTATGGAGAGTTTTCCTTTGATGGTTGGG
ATTGTTTGTAGTGGCTTGTTTCTTGTGTTTCCAAATACTC
GTTATGGTATTGGATGCATGGCAACTTAA
DMP8-like CDS Sequence (Inducer K)
SEQ ID NO: 4
ATGGACACACCAGAAGGAATCGGAATCAAAATCTACGACA
CAACCCCACAAGACGACATAGAATCATCCTCACTACCTTA
CCACCCTCCATCCACGGCAGTTCCAGGACAAGGTCGAAAG
CGACGCGCCGTCGCAAAGGGCGTGCAAAAGACGCTCTCAA
AAACATCAATGCTGGCAAATTTTCTCCCAACGGGAACCCT
TCTGACTTTCGAGATGGTTCTTCCGTCAATCTACGGTAAC
GGCGAGTGTTCCCCTGTTTCAACCACCATGATGCACGTCT
TATTGGGTCTCTGTTCTCTCTCCTGCTTCTTCTTCCATTT
CACCGACAGTTTCCGGGGACCGGACGGGAAGGTGTATTAC
GGGTTTGTTACCACTAAGGGTTTGGCTGTGTTTAAGCCTG
GATTGGATGTTGAAGTTCCCAAAGATGAGAGGTATAAATT
GGGTGTTACTGATTTTGTTCATGCTCTTATGTCTGTTTTG
GTTTTCACGGCGATCGCTTTTTCCGATCACCGGATAACTG
ACTGTGTTTTTCCTGGACATGAGAAAGCTATGGATGAAGT
TATGGAGAGTTTTCCTTTGATGGTTGGGATTGTTTGTAGT
GGCTTGTTTCTTGTGTTTCCAAATACTCGTTATGGTATTG
GATGCATGGCTACTTAA
DMP8-like CDS Sequence (Inducer C)
SEQ ID NO: 5
ATGGACACACCAGAAGGAATCGGAATCAAAATCTACGACA
CAACCCCACAAGACGACATAGAATCATCCTCACTACCTTA
CCACCCTGATCAGCCTCCATCCACGGCAGTTCCGGGACAA
GGTCGAAAGCGACGCGCCGTCGCAAAGGGCGTGCAAAAGA
CGCTCTCAAAAACATCAATGCTCGCAAATTTTCTCCCAAC
GGGAACCCTTCTGACTTTCGAGATGGTTCTTCCGTCAATC
TACGGTAACGGCGAGTGTTCCCCTGTTTCAACCACCATGA
TGCACGTCTTATTGGGTCTCTGTTCTCTCTCCTGCTTCTT
CTTCCATTTCACCGACAGTTTCCGGGGACCGGACGGGAAG
GTGTATTATGGGTTTGTTACCACTAAGGGTTTGGCTGTGT
TTAAGCCTGGATTGGATGTTGAAGTTCCCAAAGATGAGAG
GTATAAATTGGGTGTTACTGATTTTGTTCATGCTCTTATG
TCTGTTTTGGTTTTCACGGCGATCGCTTTTTCCGATCACC
GGATAACTGACTGTGTTTTTCCTGGACATGAGAAAGCTAT
GGATGAAGTTATGGAGAGTTTTCCTTTGATGGTTGGGATT
GTTTGTAGTGGCTTGTTTCTTGTGTTTCCAAATACTCGTT
ATGGTATTGGATGCATGGCTACTTAA

1. A method of producing a Cannabis haploid/di-haploid plant in a single generation, the method comprising:
(a) obtaining pollen comprising a mutation in DMP8;
(b) using the pollen to pollinate a Cannabis plant;
(c) collecting a seed from the pollenated Cannabis plant;
(d) identifying putative haploid Cannabis plants from the seed; and
(e) producing a Cannabis haploid/di-haploid plant in a single generation.

2. The method of claim 1, wherein the mutation in DMP8 results in a deletion of a single amino acid in the amino terminus of DMP8.

3. The method of claim 2, wherein the mutation in DMP8 is encoded by the nucleic acid sequence of SEQ ID NO: 4 or SEQ ID NO: 5.

4. The method of claim 1, wherein the Cannabis plant is selected from the group consisting of Cannabis sativa, Cannabis indica, and Cannabis ruderalis.

5. The method of claim 1, wherein the Cannabis haploid/di-haploid plant produced is identified by a phenotypic indicator.

6. The method of claim 5, wherein the phenotypic indicator is selected from the group consisting of dwarfism, ectopic hair/trichome presence on leaves, and a different leaf structure.

7. The method of claim 6, wherein the different leaf structure comprises leaves with 3 leaflets with 7 or 8 lobes in the central and lateral leaflets.

8. The method of claim 7, further comprising the step of confirming the putative haploid Cannabis plant utilizing genetic techniques.

9. The method of claim 8, wherein the genetic technique is selected from chromosome counting, seed sterility, and single nucleotide polymorphism (SNP) genetic analysis.

10. The method of claim 9, wherein the chromosome counting is performed by flow cytometry.

11. The method of claim 9, wherein the SNP genetic analysis is confirmed via DNA sequencing.

12. The method of claim 1, wherein the Cannabis haploid/di-haploid plant does not comprise the mutation in DMP8.

13. A Cannabis haploid/di-haploid plant produced by the method of claim 1.

14. The Cannabis haploid/di-haploid plant of claim 13, where the Cannabis haploid/di-haploid plant comprises cells, tissues, and organs.

15. The Cannabis haploid/di-haploid plant of claim 14, wherein the organs include seeds, leaves, fruits, flowers, stems, and/or roots.

16. The Cannabis haploid/di-haploid plant of claim 13, wherein the Cannabis haploid/di-haploid plant includes propagation materials selected from the group consisting of pollen, ovaries, ovules, germs, endosperms, egg cells, cleavage, roots, root tips, hypotcotyls, cotyledons, stems, leaves, flowers, anthers, seeds, meristematic cells, protoplasts, and tissue cultures.

17. The method of claim 1, further comprising:
(f) obtaining a seed from the produced Cannabis haploid/di-haploid plant.

18. The method of claim 17, further comprising:
(g) growing a Cannabis haploid/di-haploid plant from the seed.

19. A Cannabis haploid/di-haploid seed obtained by the method of claim 17.

20. The Cannabis haploid/di-haploid seed of claim 19, wherein the seed is genetically identical with the pollenated Cannabis plant.
